SEQUENCE LISTING (8 sequences)

SEQ ID NO: 1 - DNA, 20 bp, Artificial Sequence
  Description of Artificial Sequence Synthetic primer
tggtgcctggtctgatgatg

SEQ ID NO: 2 - DNA, 21 bp, Artificial Sequence
  Description of Artificial Sequence Synthetic primer
gtggtaaccgctcaggtgttg

SEQ ID NO: 3 - DNA, 21 bp, Artificial Sequence
  Description of Artificial Sequence Synthetic primer
ccctcctgatcttgtgttgga

SEQ ID NO: 4 - DNA, 19 bp, Artificial Sequence
  Description of Artificial Sequence Synthetic primer
tcaacccgagctcctggaa

SEQ ID NO: 5 - DNA, 25 bp, Artificial Sequence
  Description of Artificial Sequence Synthetic primer
caaccaacaagtgatattctccatg

SEQ ID NO: 6 - DNA, 21 bp, Artificial Sequence
  Description of Artificial Sequence Synthetic primer
gatccacactctccagctgca

SEQ ID NO: 7 - DNA, 23 bp, Artificial Sequence
  Description of Artificial Sequence Synthetic primer
aaccgtgaaaagatgacccagat

SEQ ID NO: 8 - DNA, 20 bp, Artificial Sequence
  Description of Artificial Sequence Synthetic primer
cacagcctggatggctacgt

(19) United States
(12) Patent Application Publication
(10) Pub. No.: US 2008/0145465 A1
(43) Pub. Date: Jun. 19, 2008

(54) Sesquiterpene lactone extract from artemisia leucodes for reducing inflammation and down-regulating pro-inflammatory gene expression

(76) Inventors: Barbara M. Schmidt, Somerset, NJ (US); Igor V. Belolipov, Tashkent (UZ); Anvar Kurmukov, Tashkent (UZ); Salakhutdin Zakirov, Tashkent (UZ); Ilya Raskin, Manalapan, NJ (US)

Correspondence Address: MATHEWS, SHEPHERD, MCKAY, & BRUNEAU, P.A., 29 THANET ROAD, SUITE 201, PRINCETON, NJ, 08540, US

(21) Appl. No.: 11/978,704

(22) Filed: Oct. 29, 2007

Related U.S. Application Data
(60) Provisional application No. 60/855,328, filed on Oct. 30, 2006.

(51) Int. Cl. A61K 36/282; A61P 9/00; A61P 43/00; A61P 19/02; A61P 17/00

(52) U.S. Cl. 424/764

(57) ABSTRACT

The sesquiterpene lactone extract from Artemisia leucodes is a potent anti-inflammatory agent in vivo. Methods of obtaining the anti-inflammatory extract and methods of treating inflammation with the extract are disclosed. A product comprising the Artemisia leucodes total sesquiterpene lactone extract is useful for treatment of inflammation.

### CROSS REFERENCE TO RELATED APPLICATION

This application claims the benefit of U.S. Provisional Patent Application No. 60/855,328, filed Oct. 30, 2006, the entirety of which is hereby incorporated by reference into this application.

### BACKGROUND OF THE INVENTION

1. Field of the Invention

The present invention relates to a sesquiterpene lactone extract from Artemisia leucodes. More specifically, the present invention relates to a method of extracting total sesquiterpene lactones from Artemisia leucodes and use of the lactones for reducing inflammation and down regulating inflammatory gene expression.

2. Description of Related Art

Artemisia leucodes Schrenk is a wild species of the Asteraceae family native to the mountains of central Asia. There are over 2000 different Artemisia species with common names including mugwort, sage, sagebrush, wormwood, and tarragon. Medicinal uses for Artemisia species are diverse including a revolutionary treatment for multi-drug resistant strains of malaria developed from Artemisia annua as described in Sriram et al., Progress in the research of artemisinin and its analogues as antimalarials: an update, Natural Products Research 18:503-527 (2004). A. leucodes is rich in sesquiterpene lactones, one of which has been tested and shown to be beneficial for inflammation. Kurmukov, A. G., Anti-inflammatory activity of the lactone leukomisin. Meditsinskii Zhurnal Uzbekistana 9:72-75 (1987). Sesquiterpene lactones are C₁₅ terpenoid compounds that have a range of biological and pharmaceutical activities. They have been reported as the active compounds of some well-known medicinal plants, such as Arnica montana (leopard's bane) and Tanacetum parthenium (feverfew) and have been used clinically for migraines and arthritis. Studies have shown that sesquiterpene lactones inhibit pro-inflammatory gene expression through inactivation of the transcription factor nuclear factor-κB (NF-κB). Several pro-inflammatory genes, including those coding for cyclooxygenase-2 (COX-2), tumor necrosis factor-alpha (TNF-α) inducible nitric oxide synthase (iNOS), and interleukin 1, beta (Il1β.) contain a binding site in their promoter region for NF-κB and therefore their expression can be mediated through the NF-κB pathway.

Inflammation plays an important role in the development of various diseases such as cancer, rheumatoid arthritis and arteriosclerosis. Inflammatory diseases are currently treated with steroidal and nonsteroidal anti-inflammatory drugs (NSAIDs). Unfortunately, both of these widely-prescribed drug classes have significant negative side effects, reducing their use in certain segments of the population. There is a need to develop new drugs with novel modes of action that do not produce considerable side effects. The uses of plant extracts as anti-inflammatory therapeutics are widely reported and can provide safe, efficacious, and cost-effective alternatives to synthetic drugs.

There are several sesquiterpene lactones from A. leucodes believed to have biological activity. Leukomisin, also known as deacetoxymatricarin, has demonstrated anti-inflammatory effects in mice and rats (Kurmukov 1987) apparently mediated by inhibition of pro-inflammatory factors including prostaglandins. Leukomisin has also been shown to lower cholesterol in rats as described in Kamilova et al., Hypolipidemic activity and mechanism of new antiatherosclerotic herbal agents from the flora of Central Asia, Doklady Akademii Nauk Respubliki Uzbekistan 7:57-60 (1996). Austricin also known as deacetylmatricarin, is another sesquiterpene lactone from A. leucodes that has been reported to be anti-allergenic as described in Ho et al., Desacetylmatricarin, an anti-allergic component from Taraxacum platycarpum. Planta Medica 64:577-578 (1998). The total sesquiterpene extract from A. leucodes has been tested for its effect on cholepoietic processes in rat liver as described in Tursunova et al., Effect of the total sesquiterpene extract from Artemisia leucodes on cholepoietic processes in rat liver in the normal state and during experimental hepatitis, Pharmaceutical Chemistry Journal 36:91-93 (2002).

It is desirable to provide a pharmaceutical composition containing sesquiterpene extract from A. leucodes that can be used to treat inflammatory and auto-immune diseases.

### SUMMARY OF THE INVENTION

A method of obtaining a pharmaceutical, nutraceutical, functional food or topical product comprising a total extract enriched with sesquiterpene lactones from Artemisia leucodes is provided. The method includes performing an initial extraction of Artemisia leucodes in a polar solvent, such as methanol or ethanol. This extraction is repeated and the collected extracts are combined. The extract is concentrated to remove the polar solvent. The resultant extract is diluted with water and filtered, followed by partitioning, with an extraction solvent, such as a hydrocarbon solvent, such as with chloroform, to obtain the total sesquiterpene lactone extract. The extraction solvent is evaporated to yield an extract enriched in sesquiterpene lactones comprising leukomisin and austricin.

Pharmaceutical compositions comprising the total extract from Artemisia leucodes, as described above, and one or more pharmaceutically acceptable formulation agents are also encompassed by this invention. Nutraceutical compositions comprising the total extract from Artemisia leucodes, as described above, and one or more nutraceutical-acceptable formulation agents are also encompassed by this invention.

Another aspect of the present invention provides a functional food product. The functional food product includes functional food ingredients and the total sesquiterpene lactone-enriched extract from Artemisia leucodes. Another aspect of the present invention provides for a topical product comprising the total extract from Artemisia leucodes, as described above, and one or more excipient ingredients.

In one embodiment, the pharmaceutical composition, nutraceutical, functional food or topical composition, as described above, comprise an effective amount of the total sesquiterpene lactone-enriched extract from Artemisia leucodes to treat inflammatory and autoimmune diseases including but not limited to rheumatoid arthritis, asthma, inflammatory bowel disease, Crohn's disease, multiple sclerosis, psoriasis and skin rashes, chronic obstructive pulmonary disease, allergic rhinitis, cardiovascular disease, lupus, and metabolic syndrome or to prevent and treat conditions associated with inflammation, such as skin aging, for example.

In another aspect of the invention, the total sesquiterpene lactone-enriched extract from Artemisia leucodes is used to treat and reduce inflammation.

Two animal models of inflammation, rat paw edema and cotton granuloma, were used to gauge the effectiveness of the extract in vivo. The effect on pro-inflammatory gene expression (COX-2, iNOS, and IL1β) and nitric oxide production in LPS-elicited RAW macrophages was measured to determine possible modes of action.

As used herein, by “total sesquiterpene lactone extract” is meant that the starting material is treated with solvents to extract the sesquiterpene lactone components. “Total sesquiterpene lactone” is used herein to distinguish from treatments that further separate into an individual sesquiterpene lactone or subgroups of sesquiterpene lactones.

The invention will be more fully described by reference to the following drawings.

### BRIEF DESCRIPTION OF THE DRAWINGS

FIG. 1 is a drawing of the structures of two major guaianolide sesquiterpene lactones found in Artemisia leucodes, (1) leukomisin, and (2) austricin.

FIG. 2 is a LC-MS chromatogram of the sesquiterpene lactone extract from Artemisia leucodes, AL-1. Peak 1 (t_{R} 19.1)=austricin; peak 2 (t_{R}25.4)=leukomisin.

FIG. 3 shows electron impact fragmentation patterns for leukomisin (Spectrum 1A; t_{R} 25.4) and austricin (Spectrum 1B; t_{R} 19.1).

### DETAILED DESCRIPTION

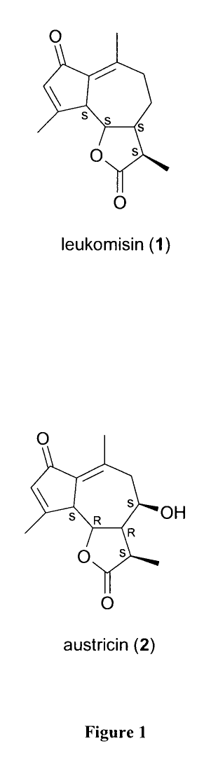

The present invention relates to a total sesquiterpene lactone extract from Artemisia leucodes prepared by an initial step of performing an initial extraction in a polar solvent to form an extract. This extraction is repeated and the collected extracts are combined. The extract is concentrated to remove the polar solvent. The resultant extract is diluted with water and filtered followed by partitioning with an extract solvent, such as a hydrocarbon solvent, such as with chloroform to obtain the total sesquiterpene lactone extract. The final extraction solvent is evaporated to yield an extract enriched in sesquiterpene lactones comprising leukomisin and austricin (FIG. 1).

Suitable polar solvents include all straight chain and branched primary alcohols and chemical derivatives thereof, provided that the additional chemical groups do not destroy the polarity of the fluid or increase the polarity of the fluid to the level of water, which is expressly excluded from the definition of a polar fluid. Preferred polar fluids are liquids, such as the lower molecular weight, straight chain, primary alcohols, such as ethanol or methanol. Suitable polar solvents also include a mixture of water and a polar fluid such as methanol, ethanol, acetone, methylisolobutyl or hexane. For example, a polar solvent can be 70%-90% alcohol.

The extractions result in an extract containing at least 20% sesquiterpene lactones at ambient temperature. More preferably, the extractions can be repeated or performed for a period of time that results in an extract containing at least 30%, at least 40%, at least 50% and most preferably, at least 60% sesquiterpene lactones at ambient temperature.

A pharmaceutical composition, nutraceutical, functional food or topical product containing the extract can be obtained by admixing or processing the total sesquiterpene lactone extract with suitable excipients, carriers or other ingredients.

Various modes of administration, including all modes known in the art, are contemplated for use in delivering the total sesquiterpene lactone-enriched extract from Artemisia leucodes to a mammal such as a human patient. Preferred modes of administration of the total extract from Artemisia leucodes include parenteral (e.g., intravenous, intramuscular and subcutaneous), oral administration, and topical administration. The total sesquiterpene lactone-enriched extract from Artemisia leucodes can be added to a pharmaceutical composition, nutraceutical, functional food and/or topical composition in any suitable amount. In one embodiment, the pharmaceutical composition, nutraceutical, functional food and/or topical composition includes the Artemisia leucodes total extract of the present invention in an amount of at least 0.5% by weight, preferably from about 1% to about 80% by weight, more preferably about 1% to about 20% by weight.

In one embodiment, the pharmaceutical compositions containing the extract of the present invention may be in any form,suitable for oral use, such as e.g., tablets, troches, lozenges, aqueous or oily suspensions, dispersible powders or granules, emulsions, hard or soft capsules, or syrups or elixirs. Compositions intended for oral use can be prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient(s) in admixture with non-toxic pharmaceutically acceptable excipients, such as inert diluents, granulating, disintegrating and lubricating agents, which are suitable for the manufacture of tablets. The tablets may be uncoated or they may be coated by known techniques to delay disintegration and absorption in the gastrointestinal tract and thereby provide a sustained action over a longer period. Formulations for oral use may also be presented as hard gelatin capsules wherein the active ingredient is mixed with an inert solid diluent, or as soft gelatin capsules wherein the active ingredients is mixed with water or an oil medium. Aqueous suspensions contain the active material in admixture with excipients suitable for the manufacture of aqueous suspensions, such as e.g., suspending agents, dispersing or wetting agents, preservatives, coloring agents, flavoring agents, and sweetening agents. Dispersible powders and granules suitable for preparation of an aqueous suspension by the addition of water provide the active ingredient(s) in admixture with a dispersing or wetting agent, suspending agent and one or more preservatives. Additional excipients, for example sweetening, flavoring and coloring agents, may also be present.

Pharmaceutically acceptable carrier preparations for parenteral administration include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The active therapeutic ingredient may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringer's dextrose, and the like. Preservatives and other additives may also be present such as, for example, antimicrobials, anti-oxidants, chelating agents, inert gases, and the like.

Also contemplated as part of the present invention is the treatment of inflammation and inflammatory or autoimmune diseases in a mammal, including a human, comprising administering an effective amount of a composition comprising the Artemisia leucodes total sesquiterpenoid lactone extract of the present invention. The diseases contemplated include but are not limited to rheumatoid arthritis, osteoarthritis, asthma, emphysema, bronchitis, inflammatory bowel disease, Crohn's disease, ulcerative colitis, multiple sclerosis, psoriasis and skin rushes, chronic obstructive pulmonary disease, allergic rhinitis, cardiovascular disease, lupus, and metabolic syndrome. Other examples of autoimmune diseases for which the treatment is contemplated are celiac disease, polymyositis/dermatomyositis, Sjogren's syndrome, scieroderma and myasthenia gravis. Further examples of contemplated diseases include tendonitis and bursitis.

Although the present invention is further described in the examples below, the scope of the present invention is not limited to that described in these examples.

### EXAMPLE 1

### Extraction Procedure

Leaves from Artemisia leucodes were dried and soaked in 95% ethanol (1:5 w/v) for 24 hours at room temperature (24° C.). This process was repeated three times and the collected extracts were combined. The ethanol was removed using a rotary evaporator to the point where the extract was 10% of the original volume. The resulting extract was diluted with water (1:1 v/v) and filtered before partitioning with chloroform (1:1 v/v). The chloroform was removed using a rotary evaporator and the resulting extract, hereupon termed AL-1, was lyophilized and stored at 4° C.

### EXAMPLE 2

### Compositional Analysis of AL-1

### LC-MS Analysis

LC-MS was employed to determine the sesquiterpene lactone content of AL-1. AL-1 was separated and analyzed with the Waters (Milford, Mass.) LC-MS Integrity™ system consisting of a solvent delivery system including a W616 pump and W600S controller, W717 plus auto-sampler, W996 PDA detector and Waters TMD Thermabeam™ electron impact (EI) single quadrupole mass detector. Data were collected & analyzed with the Waters Millennium® v. 3.2 software, linked with the 6^{th} edition of the Wiley Registry of Mass Spectral Data, containing 229,119 EI spectra of 200,500 compounds. Substances were separated on a Phenomenex® Luna C-8 reverse phase column, size 150×2 mm, particle size 3 μm, pore size 100 Å, equipped with a Phenomenex® SecurityGuard™ pre-column. The mobile phase consisted of 2 components: solvent A (0.5% ACS grade acetic acid in double distilled de-ionized water, pH 3-3.5), and solvent B (100% acetonitrile). The mobile phase flow was adjusted at 0.25 ml/min, and generally a gradient mode was used for all analyses. The gradient points were for time 0.0 min—95% A and 5% B; for time 25.0 min—5% A and 95% B; held isocratic for 2 minutes and from 27.0 min to 30.0 min.—back to initial conditions of 95% A and 5% B. A column equilibration time of 15 minutes was set between subsequent injections.

### NMR Analysis and Physical Properties

The ¹H NMR (500 MHz) and ¹³C-NMR (125 MHz) spectra (CDCl₃) were recorded on a Varian Inova 500 spectrometer (Varian Instruments, Palo Alto, Calif.), using TMS as an internal standard. Melting point was determined on a Thomas Hoover Uni-Melt 6427K10 capillary melting point apparatus (Thomas Scientific, USA) and was uncorrected. Optical rotation was measured in CHCl₃ solution on a JASCO DIP-370 digital polarimeter (Jasco Limited, UK) at 25° C.

### LC-MS and NMR Analysis Results

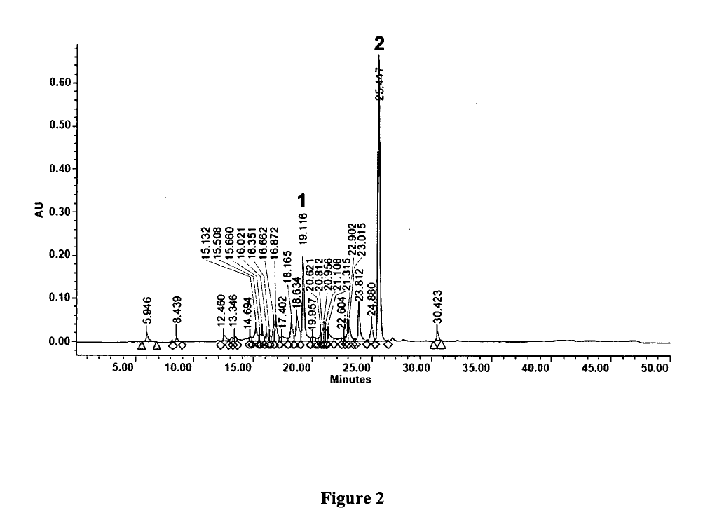

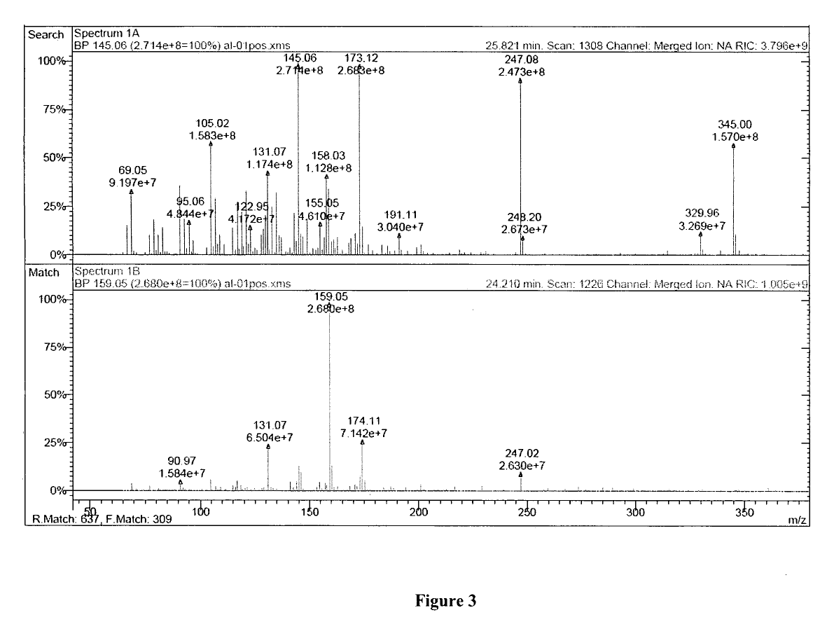

The UV-VIS chromatogram for AL-1 showed two major peaks, one at 19.1 minutes and one at 25.4 minutes, and many smaller peaks (FIG. 2). Peak t_{R} 19.1 comprised 5.5% of total peak area and peak t_{R} 25.4 comprised 84.5% of total peak area. LC-MS analysis showed the peak t_{R} 19.1 had a molecular weight of 262 with a fragmentation pattern corresponding to the sesquiterpene lactone austricin (FIG. 3, Spectrum 1A). Peak t_{R} 25.4 had a molecular weight of 246 with a fragmentation pattern corresponding to leukomisin (FIG. 3, Spectrum 1B). The retention time of a leukomisin standard was exactly the same as peak t_{R} 25.4. Since this peak comprised over 80% of the total extract, we wanted to be absolutely sure this compound was indeed leukomisin and not a stereoisomer or mixture. Therefore, the peak was isolated using preparative HPLC for further analysis of physical properties and ¹H and ¹³C NMR. The melting point and optical rotation for the isolated compound (200-204° C., +63.5) matched that of leukomisin (197-208° C., +61) (Martinez and Munoz-Zamora, 1988). ¹H and ¹³C NMR both showed the isolated peak had chemical shifts identical to those reported for leukomisin (Table 1). Considering the physical properties, LC-MS, and NMR data, the peak t_{R} 25.4 was conclusively identified as leukomisin.

TABLE 1
NMR (CDCl₃) chemical shifts for peak 2 (t_{R} 25.4)
isolated from AL-1 and those reported for leukomisin
(Martinez and Munoz-Zamora, 1988).

| | ¹H NMR (500 MHz) | | ¹³C NMR (125 MHz) | |
|---|---|---|---|---|
| position | peak | leukomisin | peak | leukomisin |
| 1 | | | 132.1 | 131.9 |
| 2 | | | 194.3 | 195.8 |
| 3 | 6.16 dq | 6.187 dq | 135.8 | 135.5 |
| 4 | | | 170.2 | 169.9 |
| 5 | 3.40 d, J = 10.0 Hz | 3.433 d, | 52.7 | 52.6 |
| | | J = 10.0 | | |
| 6 | 3.62 t, J = 10.0 Hz | 3.645 | 84.4 | 84.2 |
| | | J = 10.0 Hz | | |
| 7 | 1.96 m | 1.964 dddd | 56.6 | 56.4 |
| 8 | 1.35 dddd, J = 12.5, | 1.380 dddd, | 26.2 | 26.0 |
| | 1.6 Hz, 1.96 m | 2.040 m | | |
| 9 | 2.34 dd, J = 6.9, | 2.370 ddd, | 37.7 | 37.6 |
| | 1.9 Hz | 2.445 ddd | | |
| 10 | | | 152.4 | 152.1 |
| 11 | 2.24 dq | 2.275 dq | 41.3 | 41.1 |
| 12 | | | 177.9 | 177.5 |
| 13 | 1.26 d, J = 7.0 Hz | 1.296 d | 12.5 | 12.3 |
| 14 | 2.43 s | 2.456 s | 21.8 | 21.6 |
| 15 | 2.29 s | 2.318 d | 20.0 | 19.8 |

Many of the smaller peaks in AL-1 also had molecular weights and fragmentation patterns corresponding to sesquiterpene lactones. However due to their low abundance, no further identification was made.

### EXAMPLE 3

### Statistical Analysis

All data are expressed as means±SE. One-way ANOVA (analysis of variance) was used to determine the significance of treatments in animal studies. Student's t-test was carried out to determine the significance of difference between control and treatments in the COX enzyme and Griess assay. Treatments were considered significantly different if p<0.05.

### EXAMPLE 4

### Cell Assays

### Cell Culture

RAW 264.7 murine monocyte/macrophages (ATCC TIB-71) were maintained in Dulbecco's Modified Eagle Medium (D-MEM, Invitrogen Corp., Carlsbad, Calif.) supplemented with 10% fetal bovine serum (FBS, Invitrogen Corp., Carlsbad, Calif.) and 1% streptomycin and were kept in a humidified 37° C. incubator with 5% CO₂. Cells were subcultured by scraping when plates reached 90% confluency with a 1:5 ratio.

### Quantitative Real-Time Reverse Transcription Polymerase Chain Reaction (RT-PCR)

Murine RAW macrophages 264.7 were plated at a density of 0.4×10⁶ cells per well in 24-well plates 12 hours prior to treatment. A stock solution of AL-1 was created by completely dissolving the dry extract as in Example 1 in 95% ethanol. Two hours before elicitation with lipopolysaccharide (LPS, 1 μg/ml, Sigma, St. Louis, Mo.) the cells were treated with various concentrations of AL-1 (25-200 μg/ml) or vehicle control, maintaining a final concentration of 0.5% ethanol for all treatments. After six hours of treatment, RNA was extracted from cells using TRIzol® reagent (Invitrogen Corp., Carlsbad, Calif.) according to the manufacturer instructions. RNAs were quantified spectrophotometrically at 260 nm and stored at −80° C. until real-time PCR could be performed.

RNA was purified by treating with DnaseI (Invitrogen Corp., Carlsbad, Calif.) before performing reverse transcription with Stratascript Reverse Transcriptase, an RNA dependent DNA polymerase (Stratagene, La Jolla, Calif.) according to the manufacturer instructions. The cDNAs obtained were then amplified by real-time PCR. Expression of COX-2, iNOS, IL1β and β-actin gene expression levels was quantified using a Stratagene Mx 3000P™ Real-Time PCR System (Stratagene, La Jolla, Calif.). Primers for each gene were designed using Primer Express® version 2.0 software (Applied Biosystems, Foster City, Calif.; Table 2) (Giulietti et al., 2001; Overbergh et al., 2003).

TABLE 2

| Gene | | |
|---|---|---|
| (accession number) | Forward | Reverse |
| COX-2 | 5′-TGGTGCCTGGTCTGATGATG-3′ | 5′-GTGGTAACCGCTCAGGTGTTG-3 |
| (NM_011198) | SEQ ID NO:1 | SEQ ID NO:2 |
| iNOS2 | 5′-CCCTCCTGATCTTGTGTTGGA-3 | 5′-TCAACCCGAGCTCCTGGAA-3 |
| (XM_147149) | SEQ ID NO:3 | SEQ ID NO:4 |
| 1L1β | 5′-CAACCA ACAAGTGATATTCTCCATG-3′ | 5′-GATCCACACTCTCCA GCTGCA-3′ |
| (NM_008361) | SEQ ID NO:5 | SEQ ID NO:6 |
| Actin | 5′-AACCGTGAAAAGATGACCCAGAT-3′ | 5′-CACAGCCTGGATGGCTACGT-3′ |
| (NM_007393) | SEQ ID NO:7 | SEQ ID NO:8 |

Real-time PCR analyses were carried out using a Brilliant® SYBR® Green PCR master mix kit (Stratagene, La Jolla, Calif.) according to kit instructions. Samples were amplified using the following program: two minutes incubation at 50° C.; initial denaturation and polymerase activation at 95° C. for ten minutes; 40 PCR cycles consisting of 15 seconds at 95° C. and 60 seconds at 60° C. each. The RNA expression of the target genes were analyzed by ΔΔC_{T} methods (Winer et al., 1999) using β-Actin gene as normalizer. The ΔΔC_{T} values obtained from these analyses directly reflect the relative mRNA quantities for the marker gene in response to specific treatments. Lower ΔΔC_{T} values indicate greater anti-inflammatory activity. The changes in gene expression as reflected by ΔΔC_{T} values can also be expressed as percentage genetic inhibition [(1−ΔΔC_{T})×100] indicating anti-inflammatory properties of the test compounds. Amplification of specific transcripts was further confirmed by obtaining melting curve profiles. All samples were run in duplicate; four independent analyses were performed.

### Griess Assay for NO Production

Down-regulation of various pro-inflammatory genes including iNOS, IL1β, and TNF-α can reduce nitric oxide production by macrophages. In order to confirm that changes in pro-inflammatory gene expression were affecting cellular function, the Griess assay was performed to indirectly measure nitric oxide production by LPS-induced macrophages by measuring nitrite (Misko et al., 1993). For the assay, RAW cells were plated in a 24-well plate at a minimum density of 0.4×10⁶ cells/well and grown for 24 hours. A stock solution of AL-1 was created by completely dissolving dry extract as in Example 1 in 95% ethanol. Cells were stimulated with 1 μg/mL LPS with simultaneous addition of AL-1 (10-100 μg/mL), aspirin (10 mM), or vehicle control (0.5% ethanol), maintaining a final concentration of 0.5% ethanol for every treatment. After 24 hours, conditioned media (50 μl) was removed and immediately mixed with 100 μl of Griess reagent (10% sulfanilamide, 1% naphthalene-ethylenediamine dihydrochloride in 5% H₃PO₄, all from Sigma, St. Louis, Mo.). After incubation for 15 minutes at room temperature, the samples were read at 550 nm using a microplate spectrophotometer (Molecular Devices, Sunnyvale, Calif.). Data for each treatment were normalized based on results from an MTS cell proliferation assay.

### MTS Cell Proliferation Assay

A CellTiter 96 assay kit (Promega Corp. Madison, Wis.) was used to determine the relative number of viable cells remaining after incubation with treatments. For the assay, old media containing treatments or controls was removed from the 96-well plates and replaced with 100 μL fresh media per well. Next, 20 μL MTS/PMS reagent ([3-(4,5-dimethylthiazol-2-yl)-5-(3-carboxymethoxyphenyl)-2-(4-sulfophenyl)-2H-tetrazolium plus phenazine methosulfate) was added to each well followed by a three hour incubation period at 37° C., 5% CO₂. Plates were read using a microplate reader (Molecular Devices, Sunnyvale, Calif.) at 490 nm. A blank row corrected for background noise and data was expressed as a percent of the LPS control. Each treatment was tested on three different plates with 8 wells on each plate for a total of 24 wells.

### Results

### Gene Expression and Nitric Oxide Production

AL-1 and leukomisin were tested to see if they affect regulation of pro-inflammatory genes in macrophages elicited with LPS. AL-1 down-regulated COX-2, iNOS, and IL1β in a dose dependent manner (IC₅₀=62.14, 56.16, and 53.48 μg/mL, respectively) (Table 3). In contrast, leukomisin had no affect on gene expression (Table 4).

TABLE 3
Pro-inflammatory gene expression in RAW cells elicited
with LPS and treated with AL-1. Values are percent
inhibition of gene expression ± SE.

| Concentration (μg/ml) | COX-2 | iNOS | IL1β |
|---|---|---|---|
| 3.125 | −37.5 ± 12.6 | −18.3 ± 19.6 | −29.9 ± 13.3 |
| 6.250 | −17.3 ± 15.3 | −15.8 ± 25.9 | −3.5 ± 13.6 |
| 25.00 | −3.3 ± 9.1 | 2.8 ± 13.6 | 2.5 ± 18.6 |
| 50.00 | 45.3 ± 1.8 | 59.3 ± 6.9 | 72.3 ± 7.5 |
| 100.0 | 95.3 ± 1.0 | 99.3 ± 0.3 | 99.8 ± 0.3 |

TABLE 4
Pro-inflammatory gene expression in RAW cells elicited with
LPS and treated with AL-1, leukomisin, or austricin. Values
less than 100 represent inhibition of gene expression.

| | COX-2 | iNOS | IL1β |
|---|---|---|---|
| LPS control | 100 | 100 | 100 |
| AL-1 100 μg | 33 | 46 | 45 |
| AL-1 50 μg | 65 | 91 | 52 |
| AL-1 25 μg | 88 | 123 | 83 |
| leukomisin 75 μg | 159 | 126 | 281 |
| leukomisin 50 μg | 165 | 151 | 161 |
| leukomisin 25 μg | 140 | 127 | 110 |
| austricin 75 μg | 102 | 113 | 65 |
| austricin 50 μg | 116 | 108 | 65 |
| austricin 25 μg | 120 | 102 | 58 |

In a similar experiment, AL-1, leukomisin and austricin were tested in gene expression assays. AL-1 was more active than either of the pure compounds, leukomisin or austricin (Table 4). AL-1 reduced expression of all three genes tested, whereas leukomisin had no activity and austricin had only marginal activity reducing expression of IL1β. The activity of austricin was much lower than would be expected for a pure compound.

In the in vitro work with RAW macrophages, leukomisin had no effect on the expression of three pro-inflammatory genes (COX-2, iNOS, IL1β), one of which (COX-2) is directly involved in the production of prostaglandins. The results of these experiments are surprising considering that AL-1 contains approximately 85% leukomisin, and that other experimenters reported that leukomisin had anti-inflammatory effects in rat and mice models including the rat paw edema and cotton granuloma test (Kurmukov, 1987).

Both leukomisin and AL-1 inhibited NO production from RAW macrophages elicited with LPS [Table 5; IC₅₀=160 μg/mL (650 μM) and 105 μg/mL, respectively]. Austricin had a negligible effect on NO production [IC₅₀=440 μg/mL (1.68 mM)].

Aspirin was used as a positive control to ensure assay reproducibility. At 10 μM, aspirin inhibited NO production to 69%±1.9 of control.

TABLE 5
Effect of AL-1, leukomisin, austricin, and aspirin on nitric oxide (NO)
production from RAW macrophages as measured by the Griess assay.

| Treatment | 100 μg/ml | 50 μg/ml | 20 μg/ml | 4 μg/ml | 2 μg/ml | IC₅₀ |
|---|---|---|---|---|---|---|
| AL-1 | 55 ± 2* | 56 ± 1* | 58 ± 4* | 88 ± 4* | 91 ± 1 | 105 μg/ml |
| leukomisin | 64 ± 2* | 72 ± 1* | 82 ± 4* | 98 ± 1 | 100 ± 2 | 160 μg/ml |
| austricin | 84 ± 2* | 89 ± 1* | 89 ± 1* | 100 ± 1 | 100 ± 1 | 440 μg/ml |

Values are means ± SE;
n = 8;
*p ≦ 0.05;
significance of any treatment was determined with respect to untreated control.

Down regulating pro-inflammatory gene expression can be an effective target for treating inflammatory conditions like arthritis. Testing on cells showed the ability of AL-1 to affect three genes involved in key aspects of inflammation. IL-1β is a cytokine that acts as a signaling molecule for immune cells to coordinate the inflammatory response. COX-2 has been widely publicized as a popular target for many modern anti-inflammatory drugs. It is an enzyme necessary for the formation of prostaglandins, pro-inflammatory eicosanoids. COX-1 is constitutively expressed in many cells, but COX-2 expression is stimulated by cytokines, growth factors, and endotoxins like LPS. iNOS is an enzyme produced by macrophages in response to cytokines and other immune response signaling compounds. This enzyme produces nitric oxide, which acts as a signaling molecule in the inflammatory response. In LPS-elicited macrophages, AL-1 down regulated all three genes in a dose-dependent manner (Table 3) and AL-1, leukomisin, and austricin reduced production of nitric oxide in a dose-dependent manner (Table 5). AL-1 (IC₅₀=105 μg/mL) had higher inhibitory activity than either leukomisin [IC₅₀=160 μg/ml (650 μM)] or austricin [IC₅₀=440 μg/ml (1.68 mM)], suggesting a potentiating effect with a mixture of compounds. Still, the inhibitory effect on nitric oxide production does not explain the potent anti-inflammatory effect observed in rats, especially considering aspirin at 10 μM reduced NO production to 69% of control in the Griess assay and was overall less active than AL-1 in both of the animal experiments. Therefore, it is likely this mechanism is also not fully responsible for the observed in vivo activity.

### EXAMPLE 5

### Carrageenan-Induced Rat Paw Edema

### Methods

Groups of five adult male and female Wistar rats with body weights from 136 to 170 g were used for this study. Rats were housed five animals per cage in a room kept at 24-26° C. and fed ad libitum. Before the experiment began, rat paw sizes were recorded by measuring paw volume three times on each animal using a plethysmometer (Ugo Basile, Comerio VA, Italy). The values were averaged to give a baseline paw size value. To start the experiment, animals were given a subcutaneous injection of 1% carrageenan (100 μl; Sigma, St. Louis, Mo.) in the area of the back paw to induce an acute inflammatory reaction (paw edema). One hour after carrageenan injection, rats were orally gavaged with AL-1, aspirin, or vehicle control (5% ethanol). Paw size was measured at three hours and five hours after injection. The increase in paw edema at each time point was determined by comparison with paw volume measured pre-injection. Inhibition of edema was calculated by comparison with vehicle control at the same time point.

### Results

The carageenan-induced rat paw edema model measures the ability of anti-inflammatory agents to reduce acute swelling induced by injecting a carrageenan solution into the hind paw. As shown in Table 2, AL-1 at 50 mg/kg significantly reduced paw edema by 60% three hours after injection with carrageenan. When the dose was increased to 100 and 200 mg/kg the anti-inflammatory effect was enhanced accordingly to 71% and 80% of control. In five hours after carrageenan, the effect of AL-1 increased to 69, 81 and 79% of control according to the injected doses. By comparison, aspirin (200 mg/kg) reduced inflammation by 53% after three hours and 56% after five hours.

Artemisia leucodes is rich in sesquiterpene lactones with anti-inflammatory activity. The whole sesquiterpene extract AL-1 significantly reduced inflammation in the rat paw edema model, with greater potency than aspirin (Table 6). Since AL-1 was orally gavaged, these studies prove AL-1 is both bioavailable and pharmacologically active against inflammation. In the rat edema model, a 200 mg/kg dose of AL-1 was more effective at reducing paw swelling than aspirin. Considering aspirin is a pure compound, AL-1 had a very potent anti-inflammatory effect. AL-1 contains several sesquiterpene lactones that potentially work synergistically producing a greater anti-inflammatory effect than one pure compound.

TABLE 6
Effect of AL-1 and aspirin orally administered to rats 1 h before
injection of 1% carrageenan in the hind paw. Increases in paw volumes
were measured 3 and 5 h; percent increase was calculated compared to
basal paw volume measured before the experiment. Values are means ±
SE; n = 5; *p ≦ 0.05; significance of any treatment was determined
with respect to untreated control.

| | aspirin | AL-1 | AL-1 | AL-1 |
|---|---|---|---|---|
| | 200 mg/kg | 50 mg/kg | 100 mg/kg | 200 mg/kg |
| 3 h | 53 ± 1* | 60 ± 7* | 71 ± 6* | 80 ± 5* |
| 5 h | 56 ± 1* | 69 ± 4* | 81 ± 5* | 80 ± 8* |

### EXAMPLE 6

### Cotton Granuloma Test

### Methods

Male white rats bred from Wistar weighing 175-195 g were housed in a controlled atmosphere at 23-24° C. and 60-70% humidity with a 12 hour light/dark cycle for one week prior to testing. Free access to standard rat chow and water was provided throughout the test. Three sterile cotton pellets (10 mg) were implanted hypodermically into the groin and abdominal area of rats under Nembutal (Sigma, St. Louis, Mo.) narcosis (40 mg/kg intraperitoneally). AL-1, aspirin (Sigma, St. Louis, Mo.), and vehicle control (5% ethanol) were administered by oral gavage one day before implantation, the day of implantation (1 hour before operation), and subsequently for six days after the surgery, once per day. On the seventh day after surgery, rats were euthanized by means of a large dose of Nembutal (70 mg/kg). Cotton pellets were removed and fresh weights were recorded. The cotton pellets were then were placed into an oven (60° C.) for 24 hours and weighed again.

### Results

The cotton granuloma test measures chronic inflammation by introducing a foreign irritant (cotton pellet) into the abdominal cavity of a rat. The rat immune system infiltrates the area in and around the cotton pellet and eventually forms a granuloma of lymphocytes. When rats received AL-1 (50 mg/kg) one day before and six days after cotton pellet implantation, the resulting infiltrate and granuloma were inhibited by 38 and 59%, respectively (Table 7). When rats received aspirin (200 mg/kg), infiltrate and granuloma were reduced by 34 and 44% respectively.

TABLE 7
Effect of orally administered AL-1 and aspirin on inflammation caused
by 3 cotton pellets (10 mg) surgically implanted in rat abdominal cavities.
Infiltrate corresponds to the wet weight of pellet; granuloma corresponds
to the dry weight of pellet. Values are means of average pellet weight
from 5 animals ± SE; *p ≦ 0.05.

| | vehicle | aspirin | AL-1 |
|---|---|---|---|
| | control | 200 mg/kg | 50 mg/kg |
| infiltrate | 139 ± 10 mg | 92 ± 12 mg* | 86 ± 16 mg* |
| granuloma | 38 ± 7 mg | 21 ± 3 mg* | 16 ± 1 mg* |

The whole sesquiterpene extract AL-1 significantly reduced inflammation in the cotton granuloma model. In comparison to the rat paw edema model, the cotton granuloma model measures a more chronic type of inflammatory reaction to the presence of a foreign object, in this case a sterile cotton pellet. AL-1 was gavaged for 6 days while the cotton pellets were inside the rat abdominal and chest cavity. When the pellets were removed at the end of the study, there was a significant reduction in infiltrate to the cotton pellet and an even greater reduction in granuloma formation (Table 7). AL-1 was more potent than aspirin, indicating that a mixture of several anti-inflammatory compounds with different modes of action is more effective than one single compound in reducing inflammation.

The test results establish that AL-1, the whole sesquiterpene extract from Artemisia leucodes, is bioavailable and pharmacologically active against inflammation in vivo, reducing swelling in the rat paw edema assay and reducing a more chronic inflammatory response induced by implantation of cotton pellets into the abdominal cavities of rats.

### EXAMPLE 7

### Colorimetric COX Inhibitor Screening Assay

### Methods

An ovine COX inhibitor screening assay (Cayman Chemical Company, Ann Arbor, Mich.) was used to measure inhibition of purified COX-1 and COX-2. This assay measures the peroxidase component of cyclooxygenases colorimetrically by monitoring the appearance of oxidized N,N,N′,N′-tetramethyl-p-phenylenediamine (TMPD) at 590 nm. Tablets of Vioxx (rofecoxib; Merck & Co., Inc., Whitehouse Station, N.J.) were ground and dissolved in methanol (1 mg/mL) to use as a positive control. AL-1, leukomisin, and austricin were also dissolved in methanol at 1 mg/mL. The assay was performed following manufacturer instructions. Vioxx, vehicle (100% initial activity), or treatments were added to a 96-well plate along with 180 μL assay mixture provided by the manufacturer containing 150 μL assay buffer, 10 μL heme, and 10 μL enzyme (COX-1 or COX-2). A background correction row received no enzyme and 10 μL additional assay buffer. After 5 min incubation at room temp, 20 μL of a colorimetric substrate solution containing TMPD and 20 μL arachadonic acid diluted in assay buffer were added to the plate. The plate was incubated for 5 min and the absorbance was read at 590 nm using a microplate reader (Molecular Devices, Sunnyvale, Calif.). To calculate percent inhibition, average absorbance was calculated for each sample (n=3). The background blank well absorbance was subtracted from each sample. Each treatment was subtracted from the 100% initial activity sample, then divided by the 100% initial activity sample, and multiplied by 100 to give percent inhibition.

[(100% Initial activity-Blank)−(Treatment-Blank)/(100% Initial activity-Blank)]*100

The assay was repeated three separate times to confirm activity.

### Results

AL-1, leukomisin, and austricin were each tested for COX-1 and COX-2 enzyme inhibition using a colorimetric COX Inhibitor screening assay kit (Cayman Chemical Company, Ann Arbor, Mich.). The Vioxx used as a positive control was mixed with excipient and therefore is not equal to activity of pure rofecoxib measured in other studies; it was only used as a means to verify reproducible enzyme activity. IC₅₀ values for COX-2 inhibition for AL-1, leukomisin, and austricin were 141 μg/ml, 129 μg/ml (524 μM), and 516 μg/ml (2 mM), respectively (Table 8). The IC₅₀ for Vioxx was 40 μM. AL-1, leukomisin, austricin, and Vioxx did not significantly inhibit COX-1 enzyme activity (data not shown).

TABLE 8
Effect of AL-1, leukomisin, and austricin on COX-2 enzyme
activity. Values are means ± SE; n = 8; *p ≦ 0.05;
significance of treatment was determined with respect to
maximum (100%) enzyme activity control.

| Treatment | 225 μg/ml | 115 μg/ml | 45 μg/ml | IC₅₀ |
|---|---|---|---|---|
| AL-1 | 58 ± 2* | 46 ± 9 | 43 ± 7 | 141 μg/mL |
| leukomisin | 65 ± 5 | 51 ± 3 | 34 ± 1 | 129 μg/mL |
| austricin | 31 ± 1 | 29 ± 2 | 19 ± 1 | 516 μg/mL |

The inflammatory response is mediated through several pathways including the prostaglandin pathway involving COX-2. Reducing COX-2 activity can be an effective target for treating inflammatory conditions like arthritis. In an attempt to discover the mode of action for the anti-inflammatory activity observed in animals, we tested the ability of AL-1 and two sesquiterpene lactones isolated from AL-1, austricin and leukomisin, to affect COX enzyme activity. AL-1 and leukomisin showed moderate COX-2 inhibition (Table 8) but did not inhibit COX-1 activity (data not shown). Austricin had very little activity against COX-2 (Table 8) and did not inhibit COX-1 (data not shown). The activity of AL-1 and its sesquiterpene lactones was not as strong as other plant extracts found to be selective COX-2 inhibitors. For example, a standardized hops extract was shown to inhibit COX-2 in a whole blood assay with an IC₅₀=20.4 μg/mL (Hougee et al., 2006). An ethanolic extract of Saururus chinensis inhibited COX-2 dependent phases of prostaglandin D(2) in bone marrow-derived mast cells with an IC₅₀=14.3 μg/mL (Lee et al., 2006). The stem extract of Clematis pickeringii was shown to inhibit the activity of COX-2 with an IC₅₀=101.2 μg/mL. Even though the activity of Vioxx used as a positive control in the present experiment was not representative of pure rofecoxib due to its dilution with excipient, it was still 10-50 times more active than our pure compounds from AL-1. Since the in vitro activity of AL-1 against COX-2 enzyme activity can be described as moderate at best, this activity alone cannot explain the potent anti-inflammatory activity observed in rats.

It is to be understood that the above-described embodiments are illustrative of only a few of the many possible specific embodiments, which can represent applications of the principles of the invention. Numerous and varied other arrangements can be readily devised in accordance with these principles by those skilled in the art without departing from the spirit and scope of the invention.

What is claimed is:

1. A method of treating a subject having inflammatory or autoimmune disease comprising administering to the subject a therapeutically effective amount of a total sesquiterpene lactone extract from Artemisia leucodes.

2. The method of claim 1 wherein the administering to the subject is performed orally or topically.

3. The method of claim 1 wherein the extract contains at least 20% sesquiterpene lactones.

4. The method of claim 1 wherein the inflammatory or autoimmune disease is selected from the group consisting of rheumatoid arthritis, osteoarthritis, inflammatory bowel disease, multiple sclerosis, psoriasis and skin rashes, chronic obstructive pulmonary disease, allergic rhinitis, cardiovascular disease, lupus, and metabolic syndrome.

5. The method of claim 1 wherein the administration of said extract down-regulates the pro-inflammatory gene COX-2, iNOS, or IL1β.

6. A product for the treatment of inflammatory or autoimmune disease comprising a therapeutically effective concentration of total sesquiterpene lactone extract from Artemisia leucodes admixed with excipient and/or carrier suitable for administration as a pharmaceutical, nutraceutical, functional food or topical product.

7. The product of claim 6 wherein the extract contains at least 30% sesquiterpene lactones.

8. A method of obtaining a pharmaceutical, nutraceutical, functional food or topical product comprising a total sesquiterpene lactone extract from Artemisia leucodes comprising the steps of:
i) performing an extraction of Artemisia leucodes in a polar solvent to form an initial extract containing extracted material;
ii) adding water to the extracted material to form an aqueous extract;
iii) partitioning the aqueous extract with a hydrocarbon solvent; and
iv) removing the hydrocarbon solvent to obtain total sesquiterpene extract from Artemisia leucodes.

9. The method of claim 8 further comprising after step iv), the step of:
admixing suitable excipients and/or carriers with said total sesquiterpene extract.

10. The method of claim 8 wherein the polar solvent is ethanol or methanol.

11. The method of claim 8 wherein step i) through step iv) are repeated or performed for a period of time that result in an extract containing at least 30% sesquiterpene lactones.
